(19) United States
(12) Patent Application Publication
(10) Pub. No.: US 2011/0218215 A1
(43) Pub. Date: Sep. 8, 2011

(54) Compositions and Methods for Treating Social Anxiety

(76) Inventor: Benjamin D. Holly, Mandeville, LA (US)

(21) Appl. No.: 13/040,312

(22) Filed: Mar. 4, 2011

Related U.S. Application Data
(60) Provisional application No. 61/310,577, filed on Mar. 4, 2010.

(51) Int. Cl. A61K 31/46; A61P 25/22

(52) U.S. Cl. 514/304

(57) ABSTRACT

The disclosure provides a pharmaceutical composition for treating social anxiety, performance anxiety, and social phobia comprising a therapeutic amount for die treatment of a patient of a β-adrenergic receptor antagonist, an anti-diarrheal compound, and an optional anticholinergic compound. The β-adrenergic receptor antagonist may be the lipophilic β-blocker propranolol HCl, the anti-diarrheal compound may be the opioid diphenoxylate HCl, and the optional anticholinergic compound may be atropine sulfate. The composition for treating performance anxiety and social phobia can further include a pharmaceutically acceptable carrier. A method of preventing or treating social anxiety, performance anxiety, and social phobia in a patient is also provided, comprising administering a composition of the disclosure to a patient in need of such treatment. The composition administered in the present method comprises a therapeutic amount of a β-adrenergic receptor antagonist, an anti-diarrheal compound, and an optional anticholinergic compound.

### CROSS-REFERENCE TO RELATED APPLICATIONS

This Non-Provisional Patent Application, filed under 35 U.S.C. §111(a), claims the benefit under 35 U.S.C. §119(e)(1) of U.S. Provisional Patent Application No. 61/310,577, filed under 35 U.S.C. §111(b) on Mar. 4, 2010, and which is hereby incorporated by reference in its entirety.

### BACKGROUND

1. Field

The present disclosure is directed to the treatment of performance anxiety. More specifically the disclosure provides a composition and method for treating performance anxiety.

2. Description of Related Art

The term “social anxiety” is used commonly to describe feelings of anxiety (e.g., emotional discomfort, fear, apprehension, or worry) about social situations, interactions with others, and being evaluated or scrutinized by other people. Social anxiety is a normal part of the development of social skills in young children, and tends to lessen with age, but it may persist until adolescence or may even emerge in adulthood. The frequency and degree to which individuals experience social anxiety varies, as do the situations that trigger social anxiety. For many, overcoming social anxiety is relatively easy. For some, however, it is extremely difficult. Social anxiety reportedly affects about 20% of the U.S. population.

“Social phobia” (also called “social anxiety disorder”) is a psychopathological form of social anxiety that can result in a reduced quality of life, and reportedly affects about 2-3% of the U.S. population. Treatment for social phobia usually requires treatment by therapists, psychologists, and/or support groups, in addition to medication. In addition, there is a high degree of comorbidity with obsessive-compulsive disorder; approximately 24% of individuals diagnosed with obsessive-compulsive disorder (OCD) also suffer from social phobia. Suffering OCD and social phobia together can make both disorders more difficult to overcome.

Common adult forms of social anxiety include shyness, performance anxiety, public speaking anxiety, stage fright, timidness, etc. All of these forms of social anxiety may also assume clinical forms, i.e., become anxiety disorders.

The term “social anxiety” is also used commonly in reference to experiences such as embarrassment and shame. Some psychologists, though, draw distinctions among the different types of social discomfort, with the criterion for “anxiety” being anticipation. In other words, the anticipation of an embarrassing or shameful experience is a form of social anxiety, while the embarrassment or shame themselves are not.

Some individuals experience what is termed a “specific social phobia,” in which they fear one or a limited number of specific situations (e.g., eating or drinking in public, or public speaking). Others have a generalized form of social phobia and fear many—if not all—social interactions. Commonly, the result is complete avoidance of the dreaded social situations, although some individuals engage in subtle forms of avoidance or endure social situations with great discomfort.

Physiological components of social anxiety and social phobia commonly include (but are not limited to) sweating, blushing, urinary urgency, and fecal urgency. Cognitive or perceptual components include a fear of being watched and judged by others and of doing things that will cause embarrassment, and this fear may be intense, persistent, and chronic. The resulting outward manifestation—the behavioral component—is avoidance of the anxiety-provoking situations.

Selective serotonin reuptake inhibitors (“SSRIs”, such as PAXIL®, EFFEXOR®, and ZOLOFT®) are considered by many to be the first choice treatment for generalized social phobia. SSRIs, however, tend to exhibit a delayed onset of therapeutic effects, with full efficacy not observed until about six to eight weeks after initial treatment. They have also been reportedly associated with increased risk of suicidal ideation, although these reports remain controversial.

Before the introduction of SSRIs, anti-depressants such as monoamine oxidase inhibitors (MAOIs) were used to treat social anxiety. While the efficacy of MAOIs appears comparable or even superior to SSRIs or benzodiazepines, their usefulness for treating social phobia is limited due to dietary restrictions required, their high toxicity in overdose, and their incompatibilities with other drugs.

Benzodiazepines such as alprazolam (XANAX®) and clonazepam (KLONOPIN®) are also used to treat social anxiety, providing short-term relief of severe, disabling anxiety. While benzodiazepines are occasionally prescribed for long-term everyday outside the U.S., they present significant risks for the development of drug tolerance, dependency, and recreational abuse. Thus, benzodiazepines are considered only for patients that fail to respond to safer medications.

Some people with a form of social phobia called performance anxiety (e.g., “stage fright”) have reportedly been helped by antagonists of beta-andrenergic receptors (“beta-blockers’ or “β-blockers”), which are more commonly used to control high blood pressure.

No reports have been published where the symptom of fecal urgency as an anxiety symptom associated with public speaking was specifically targeted for treatment. Thus, there exists a need for a safe, effective medication for the treatment of both the anxiety and fecal urgency associated with performance anxiety.

### BRIEF SUMMARY

The present disclosure presents the surprising finding that the combination of a β-blocker, an anti-diarrheal compound, and an optional anticholinergic compound reduce social anxiety (and performance anxiety, in particular) more than would be predicted based on the response to each component of the combination applied separately. The present disclosure meets the aforementioned need by providing a fixed-dose combination of a β-blocker, an anti-diarrheal, and (optionally) an anticholinergic to treat symptoms of social anxiety, generally, social phobia, and symptoms of performance anxiety associated with public speaking, specifically. The combination may be provided in a single dosage form, or the β-blocker may be provided separately from the anti-diarrheal and optional anticholinergic. The β-blocker, anti-diarrheal, and anticholinergic may also be provided as a unit dose. It is anticipated that individuals with social anxiety would benefit from the compositions and methods disclosed.

Propranolol hydrochloride (“propranolol HCl”, INDERAL®) is a β-blocker that controls palpitations, tachycardia, tremor and voice quavering. Diphenoxylate hydrochloride (“diphenoxylate HCl”) is an opioid agonist used for the treatment of diarrhea, and acts by slowing intestinal contractions and peristalsis. This allows the body to consolidate intestinal contents and prolong transit time, thus allowing the intestines to draw moisture out of them at a normal or higher rate and therefore stop the formation of loose and liquid stools. Atropine sulfate is a tropane alkaloid extracted from deadly nightshade (Atropa belladonna), jimsonweed (Datura stramonium), mandrake (Mandragora officinarum) and other plants of the family Solanaceae. Atropine sulfate may be included in the compositions of the present disclosure to deter abuse.

Propranolol HCl and diphenoxylate HCl have never before been combined in a single pill, nor have they been combined with atropine sulfate. The compositions of the present disclosure provide, in a convenient form, a type of comprehensive medical treatment for anxiety symptoms that have not been previously available.

The instant disclosure provides a pharmaceutical composition for treating performance anxiety or social phobia in a mammal in need thereof, comprising a therapeutic amount of a β-adrenergic receptor antagonist, an anti-diarrheal, and (optionally) an anti-cholinergic compound. Preferably, the β-adrenergic receptor antagonist is the lipophilic β-blocker propranolol HCl. The anti-diarrheal compound of the present disclosure is preferably diphenoxylate HCl. The optional anti-cholinergic compound of the present disclosure is preferably atropine sulfate. The composition for treating performance anxiety and social phobia can further include a pharmaceutically acceptable carrier, diluent or excipient.

The present disclosure also provides a method of preventing or treating performance anxiety in a patient, the method comprising administering the composition of the present disclosure to a patient in need of such treatment. The composition administered in the present method comprises a therapeutic amount of a β-adrenergic receptor antagonist, an anti-diarrheal compound, and (optionally) an anti-cholinergic compound.

In one embodiment, the present disclosure provides a pharmaceutical composition for treating social anxiety, performance anxiety, or social phobia comprising a therapeutic amount for the treatment of a patient of a β-adrenergic receptor antagonist, an anti-diarrheal compound, and an optional anticholinergic compound. The β-adrenergic receptor antagonist may be propranolol HCl. The anti-diarrheal compound may be an opioid compound. The anti-diarrheal compound may be diphenoxylate HCl. The optional anticholinergic compound may be atropine sulfate. In one aspect, the β-adrenergic receptor antagonist may be propranolol HCl, the anti-diarrheal compound may be diphenoxylate HCl, and the optional anticholinergic compound may be atropine sulfate. In one aspect, the propranolol HCl may be in an amount of from about 5 mg to about 40 mg, the diphenoxylate HCl may be in an amount of from about 2.5 mg to about 5.0 mg, and the optional atropine sulfate may be in an amount of from about 0.025 mg to about 0.05 mg.

In one embodiment, the present disclosure provides a method of treating social anxiety in a patient comprising the step of administering a composition comprising a therapeutic amount for the treatment of a patient of a β-adrenergic receptor antagonist, an anti-diarrheal compound, and an optional anticholinergic compound to a patient in need of such treatment. In one aspect, the β-adrenergic receptor antagonist may be propranolol HCl, the anti-diarrheal compound may be diphenoxylate HCl, and the optional anticholinergic compound may be atropine sulfate.

In one embodiment, the present disclosure provides a method of preventing social anxiety in a patient comprising the step of administering a composition comprising a therapeutic amount for the treatment of a patient of a β-adrenergic receptor antagonist, an anti-diarrheal compound, and an optional anticholinergic compound to a patient in need of such treatment. In one aspect, the β-adrenergic receptor antagonist may be propranolol HCl, the anti-diarrheal compound may be diphenoxylate HCl, and the optional anticholinergic compound may be atropine sulfate.

In one embodiment, the present disclosure provides a method of treating performance anxiety in a patient comprising the step of administering a composition comprising a therapeutic amount for the treatment of a patient of a β-adrenergic receptor antagonist, an anti-diarrheal compound, and an optional anticholinergic compound to a patient in need of such treatment. In one aspect, the β-adrenergic receptor antagonist may be propranolol HCl, the anti-diarrheal compound may be diphenoxylate HCl, and the optional anticholinergic compound may be atropine sulfate.

In one embodiment, the present disclosure provides a method of preventing performance anxiety in a patient comprising the step of administering a composition comprising a therapeutic amount for the treatment of a patient of a β-adrenergic receptor antagonist, an anti-diarrheal compound, and an optional anticholinergic compound to a patient in need of such treatment. In one aspect, the β-adrenergic receptor antagonist may be propranolol HCl, the anti-diarrheal compound may be diphenoxylate HCl, and the optional anticholinergic compound may be atropine sulfate.

In one embodiment, the present disclosure provides a method of treating social phobia in a patient comprising the step of administering a composition comprising a therapeutic amount for the treatment of a patient of a β-adrenergic receptor antagonist, an anti-diarrheal compound, and an optional anticholinergic compound to a patient in need of such treatment. In one aspect, the β-adrenergic receptor antagonist may be propranolol HCl, the anti-diarrheal compound may be diphenoxylate HCl, and the optional anticholinergic compound may be atropine sulfate.

In one embodiment, the present disclosure provides a method of preventing social phobia in a patient comprising the step of administering a composition comprising a therapeutic amount for the treatment of a patient of a β-adrenergic receptor antagonist, an anti-diarrheal compound, and an optional anticholinergic compound to a patient in need of such treatment. In one aspect, the β-adrenergic receptor antagonist may be propranolol HCl, the anti-diarrheal compound may be diphenoxylate HCl, and the optional anticholinergic compound may be atropine sulfate.

In one embodiment, the present disclosure provides a method of treating social anxiety in a patient comprising the steps of: a) administering a therapeutic amount of a β-adrenergic receptor antagonist; b) administering, either before, after, or simultaneous to the administration of the β-adrenergic receptor antagonist, a therapeutic amount of an anti-diarrheal compound; and c) optionally administering, either before, after, or simultaneous to the administration of the β-adrenergic receptor antagonist, a therapeutic amount of an anticholinergic compound, to a patient in need of such treatment. The β-adrenergic receptor antagonist may be propranolol HCl, the anti-diarrheal compound may be diphenoxylate HCl, and the optional anticholinergic compound may be atropine sulfate.

In one embodiment, the present disclosure provides a method of preventing social anxiety in a patient comprising the steps of: a) administering a therapeutic amount of a β-adrenergic receptor antagonist; b) administering, either before, after, or simultaneous to the administration of the β-adrenergic receptor antagonist, a therapeutic amount of an anti-diarrheal compound; and c) optionally administering, either before, after, or simultaneous to the administration of the β-adrenergic receptor antagonist, a therapeutic amount of an anticholinergic compound, to a patient in need of such treatment. The β-adrenergic receptor antagonist may be propranolol HCl, the anti-diarrheal compound may be diphenoxylate HCl, and the optional anticholinergic compound may be atropine sulfate.

In one embodiment, the present disclosure provides a method of treating performance anxiety in a patient comprising the steps of: a) administering a therapeutic amount of a β-adrenergic receptor antagonist; b) administering, either before, after, or simultaneous to the administration of the β-adrenergic receptor antagonist, a therapeutic amount of an anti-diarrheal compound; and c) optionally administering, either before, after, or simultaneous to the administration of the β-adrenergic receptor antagonist, a therapeutic amount of an anticholinergic compound, to a patient in need of such treatment. The β-adrenergic receptor antagonist may be propranolol HCl, the anti-diarrheal compound may be diphenoxylate HCl, and the optional anticholinergic compound may be atropine sulfate.

In one embodiment, the present disclosure provides a method of preventing performance anxiety in a patient comprising the steps of: a) administering a therapeutic amount of a β-adrenergic receptor antagonist; b) administering, either before, after, or simultaneous to the administration of the β-adrenergic receptor antagonist, a therapeutic amount of an anti-diarrheal compound; and c) optionally administering, either before, after, or simultaneous to the administration of the β-adrenergic receptor antagonist, a therapeutic amount of an anticholinergic compound, to a patient in need of such treatment. The β-adrenergic receptor antagonist may be propranolol HCl, the anti-diarrheal compound may be diphenoxylate HCl, and the optional anticholinergic compound may be atropine sulfate.

In one embodiment, the present disclosure provides a method of treating social phobia in a patient comprising the steps of: a) administering a therapeutic amount of a β-adrenergic receptor antagonist; b) administering, either before, after, or simultaneous to the administration of the β-adrenergic receptor antagonist, a therapeutic amount of an anti-diarrheal compound; and c) optionally administering, either before, after, or simultaneous to the administration of the β-adrenergic receptor antagonist, a therapeutic amount of an anticholinergic compound, to a patient in need of such treatment. The β-adrenergic receptor antagonist may be propranolol HCl, the anti-diarrheal compound may be diphenoxylate HCl, and the optional anticholinergic compound may be atropine sulfate.

In one embodiment, the present disclosure provides a method of preventing social phobia in a patient comprising the steps of: a) administering a therapeutic amount of a β-adrenergic receptor antagonist; b) administering, either before, after, or simultaneous to the administration of the β-adrenergic receptor antagonist, a therapeutic amount of an anti-diarrheal compound; and c) optionally administering, either before, after, or simultaneous to the administration of the β-adrenergic receptor antagonist, a therapeutic amount of an anticholinergic compound, to a patient in need of such treatment. The β-adrenergic receptor antagonist may be propranolol HCl, the anti-diarrheal compound may be diphenoxylate HCl, and the optional anticholinergic compound may be atropine sulfate.

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments, and is not intended to be limiting. Instead, the scope of the present disclosure will be established by the appended claims,

In this specification and the appended claims, the singular forms “a, ” “an,” and “the” include plural reference unless the context clearly dictates otherwise. Unless defined otherwise, all technical and scientific terms used herein have the same meaning as commonly understood to one of ordinary skill in the art to which this disclosure belongs.

The disclosure provides a pharmaceutical composition for treating performance anxiety or social phobia in a mammal in need thereof, comprising a therapeutic amount of a β-adrenergic receptor antagonist and an anti-diarrheal compound. Preferably, said composition further comprises an optional anticholinergic compound.

The β-adrenergic receptor antagonist of the composition (hereinafter “β-blocker”) can be one that crosses the blood brain barrier. The lipophilic β-blocker propranolol HCl (INDERAL®) is one example of a β-blocker that crosses the blood brain barrier. Other examples of lipophilic β-blockers include metoprolol succinate (TOPROL-XL®), and nadolol (CORGARD®). The β-blocker can alternatively be one that does not cross the blood brain barrier. For example, atenolol (TENORMIN®) acts primarily on the peripheral nervous system. Other β-blockers include but are not limited to the following: alprenolol; bucindolol; sotalol HCl (BETAPACE®); timolol maleate (BLOCADREN®); esmolol HCl (BREVIBLOC®): carteolol HCl (CARTROL®); celiprolol (CELECTOL®); carvedilol (COREG®); betaxolol HCl (KERLONE®); penbutolol sulfate (LEVATOL®); metoprolol tartrate (LOPRESSOR®); nebivolol (NEBILET®); labetalol (NORMODYNE®); acebutolol HCl (SECTRAL®); pindolol (VISKEN®); and bisoprolol fumorate (ZEBETA®).

β-blockers, as a class, are expected to be effective in the present composition because they act through the same mechanism in the subject's nervous system. Routine methods for screening compounds for their β-blocking activity and for determining which β-blockers cross the blood-brain barrier are known in the art (Spahn-Langguth et al “Improved enantiospecific RP-HPLC assays for propranolol in plasma and urine with pronethalol as internal standard” Journal of Analytical Toxicology, 15(4):209-213, 1991; Sproat and Lopez “Around the beta-blockers, one more time, “Review DICP 25(9):962-971, 1991; and Davies, C. “Chromatography of beta-adrenergic blacking agents,” Review Journal of Chromatography 531:131-180, 1990). Thus, given the teaching of the present disclosure, it is contemplated that other β-blockers, whether presently known or later developed, are within the scope of the disclosure,

The anti-diarrheal compound of the present disclosure is preferably an opioid compound. Diphenoxylate HCl is an example of an anti-diarrheal opioid compound, as are difenoxin and diphenoxylic acid. Loperamide (IMODIUM®) is an anti-diarrheal opioid compound that does not cross the blood brain barrier, and, thus, acts exclusively in the intestines. It is expected that other anti-diarrheal opioid compounds will also be effective in the present composition, whether or not they cross the blood-brain barrier. Thus, other anti-diarrheal opioid compounds whether presently known or subsequently produced are within the scope of the present disclosure. Additionally, centrally acting anticholinergics (e.g., atropine) can also be used in the present method and composition. Routine methods for screening compounds for their anticholinergic activity and for their ability to cross the blood-brain barrier are well known (for example, see Ali-Melkkila et al. “Pharmacokinetics and related pharmacodynamics of anticholinergic drugs,” Review, Acta Anaestesiologica Scandinavica 37(7);633-642, 1993).

Thus, in an embodiment of the present composition, the β-adrenergic receptor antagonist is propranolol HCl, the anti-diarrheal compound is diphenoxylate HCl, and the optional anticholinergic compound is atropine sulfate. In one aspect of this embodiment, the propranolol HCl may be in an amount of from about 5 mg to about 40 mg, the diphenoxylate HCl may be in an amount of from about 0.25 mg to about 5.0 mg, and the optional atropine sulfate may be in an amount of from about 0.025 mg to about 0.05 mg. Thus, one of the advantages of the present composition and method is the effectiveness of the components at low dosages compared to other uses of the same compounds. Lower dosages, for example as low as 3 mg of β-blocker and as low as 0.025 mg of anticholinergic are within the scope of the disclosure once routinely determined by the skilled practitioner to be effective in a given patient. Other dosages within the disclosed range will also be effective, but may be attended by tolerable side affects that are avoided at the lower dosages.

The composition for treating social anxiety, performance anxiety, and social phobia can further include a pharmaceutically acceptable carrier. The compositions can also include other medicinal agents, pharmaceutical agents, carriers, adjuvants, diluents, etc. By “pharmaceutically acceptable” is meant a material that is not biologically or otherwise undesirable, i.e., the material may be administered to an individual along with the selected compound without causing any undesirable biological effects or interacting in a deleterious manner with any of the other components of the pharmaceutical composition in which it is contained. For example, the carrier can he cornstarch. Examples of other excipients or carriers include compositions of one or more of lactose, magnesium stearate, microcrystalline cellulose and stearic acid, povidone, dibasic calcium phosphate and sodium starch glycolate. As the preferred mode of administration is oral, it is contemplated that any carrier suitable for oral administration can be the carrier of the present composition. If other modes of administration are used, carriers will be selected using standard criteria for the administration of compositions by that mode.

Because the preferred method of administration of the present compound is oral, the composition can be contained in a gelatin capsule, tablet, liquid or powder, etc for ingestion. For oral administration, fine powders or granules may contain diluting, dispersing, and/or surface active agents, and may be presented in water or in a syrup, in capsules or sachets in the dry state, or in a nonaqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and capsules are preferred oral administration forms, and these may be coated. Actual methods of preparing such dosage forms are known, or will be apparent, to those skilled in this art; for example, see Remington's Pharmaceutical Sciences (18th Ed., E. W. Martin (ed.), Mack Publishing Co., Easton, Pa.).

A method of treating social anxiety in a patient comprising administering the composition of the disclosure to a patient in need of such treatment is also provided. As described above, the composition administered in the present method comprises a therapeutic amount of a β-adrenergic receptor antagonist, an anti-diarrheal compound, and an optional anticholinergic compound. More preferably, the method of treating social anxiety comprises administering to said patient in need of such treatment a composition in which the β-adrenergic receptor antagonist is propranolol HCl and the anti-diarrheal compound is diphenoxylate HCl. Preferably, the propranolol HCl is in an amount of from about 5 mg to about 40 mg, the diphenoxylate HCl is in an amount of from about 2.5 mg to about 5 mg, and the optional atropine sulfate is in an amount of from about 0.025 mg to about 0.05 mg. However, other amounts of the compounds can be administered in accordance with the above description of the composition of the disclosure.

The disclosure also provides a method of treating or preventing performance anxiety in a patient by administering the composition comprising a therapeutic amount of a β-adrenergic receptor antagonist, an anti-diarrheal compound to a patient in need of such preventive treatment, and optionally an anticholinergic compound. More preferably, the method of treating or preventing performance anxiety in a patient comprises administering to a patient in need of such preventive treatment the composition in which the β-adrenergic receptor antagonist is propranolol HCl, the anti-diarrheal compound is diphenoxylate HCl, and the optional anticholinergic compound is atropine sulfate. Preferably, the propranolol HCl is in an amount of from about 5 mg to about 40 mg, the diphenoxylate HCl is in an amount of from about 2.5 mg to about 5 mg, and the optional atropine sulfate is in an amount of from about 0.025 mg to about 0.05 mg. However, other amounts of the compounds can be administered in accordance with the above description of the composition of the disclosure.

The disclosure also provides a method of treating or preventing social phobia in a patient by administering the composition comprising a therapeutic amount of a β-adrenergic receptor antagonist, an anti-diarrheal compound to a patient in need of such preventive treatment, and optionally an anticholinergic compound. More preferably, the method of treating or preventing social phobia in a patient comprises administering to a patient in need of such preventive treatment the composition in which the β-adrenergic receptor antagonist is propranolol HCl, the anti-diarrheal compound is diphenoxylate HCl, and the optional anticholinergic compound is atropine sulfate. Preferably, the propranolol HCl is in an amount of from about 5 mg to about 40 mg, the diphenoxylate HCl is in an amount of from about 2.5 mg to about 5 mg, and the optional atropine sulfate is in an amount of from about 0.025 mg to about 0.05 mg. However, other amounts of the compounds can be administered in accordance with the above description of the composition of the disclosure.

Because the disclosure teaches that performance anxiety can be treated or prevented using the compositions taught herein, the disclosure also provides a method of preventing or treating performance anxiety in a patient by administering a therapeutic amount of a β-adrenergic receptor antagonist and administering, either before, after, or simultaneous to the administration of the β-adrenergic receptor blocking compound, a therapeutic amount of an anti-diarrheal compound to a patient in need of such prevention or treatment (and, optionally, an anticholinergic compound). In this method, the β-adrenergic receptor antagonist can be propranolol HCl, the anti-diarrheal compound can be diphenoxylate HCl, and the optional anticholinergic compound can be atropine sulfate. The amounts administered can be as described above for the compositions of the disclosure.

The disclosure also provides a method of preventing or treating social anxiety in a patient comprising the step(s) of administering a therapeutic amount of a β-adrenergic receptor antagonist and administering, either before, after, or simultaneous to the administration of the β-adrenergic receptor blocking compound, a therapeutic amount of an anti-diarrheal compound to a patient in need of such treatment (and, optionally, an anticholinergic compound). In this method, the β-adrenergic receptor antagonist can be propranolol HCl, the anti-diarrheal compound can be diphenoxylate HCl, and the optional anticholinergic compound can be atropine sulfate. The amounts administered can be as described above for the compositions of the disclosure.

The disclosure also provides a method of preventing or treating social phobia in a patient comprising the step(s) of administering a therapeutic amount of a β-adrenergic receptor antagonist and administering, either before, after, or simultaneous to the administration of the β-adrenergic receptor blocking compound, a therapeutic amount of an anti-diarrheal compound to a patient in need of such treatment (and, optionally, an anticholinergic compound). In this method, the β-adrenergic receptor antagonist can be propranolol HCl, the anti-diarrheal compound can be diphenoxylate HCl, and the optional anticholinergic compound can be atropine sulfate. The amounts administered can be as described above for the compositions of the disclosure.

As described above, the preferred mode of administration in the present method is oral. The dosage form administered can be selected from among the standard oral dosage forms depending on the circumstances of the patient in need of the present treatment or prevention methods. Actual methods of preparing such dosage forms are known, or will be apparent, to those skilled in this art; for example, see Remington's Pharmaceutical Sciences (18th Ed., E. W. Martin (ed.), Mack Publishing Co., Easton, Pa.).

The following examples are intended to illustrate, but not limit, the disclosure. While the protocols described are typical of those that might be used, other procedures known to those skilled in the art may be alternatively employed.

### EXAMPLE 1

### The Compositions

A pharmaceutical composition for the treatment of performance anxiety is a combination of propranolol HCl, diphenoxylate HCl, and atropine sulfate. These three drugs may be combined with carriers or excipients, for example, a cornstarch excipient in a gelatin capsule. The form of the composition can vary within the parameters known in the art and as described herein.

Propranolol HCl

Propranolol (chemical formula: C₁₆H₂₁NO₂.HCl; molecular weight: 295.8 g/mol). shown below as Formula 1, bears the IUPAC name (RS)-1-(isopropylamino)-3-(1-naphthyloxy)propan-2-ol. It is a racemic mixture of R- and S-isoforms, and is available from multiple sources.

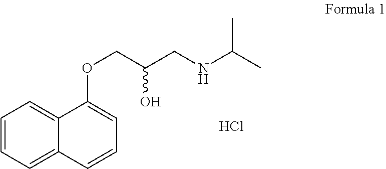

Diphenoxylate HCl

Diphenoxylate HCl (chemical formula: C₃₀H₃₂N₂O₂.HCl; molecular weight: 489.06 g/mol), shown below as Formula 2, bears the IUPAC name ethyl 1-(3-cyano-3,3-diphenyl-propyl)-4-phenyl-piperidine-4-carboxylate. It is available from multiple sources.

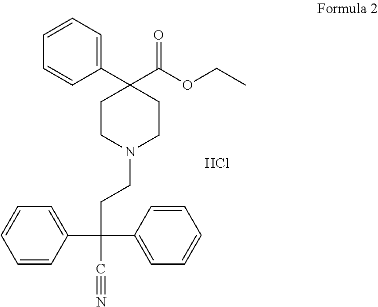

Atropine Sulfate

Atropine (chemical formula: C₇H₂₃NO₃; molecular weight: 289.369 g/mol), shown below as Formula 3, bears the IUPAC name (8-methyl-8-azabicyclo[3.2.1]oct-3-yl) 3-hydroxy-2-phenyl-propanoate. Atropine is a racemic mixture of D-hyoscyamine and L-hyoscyamine, with most of its physiological effects due to L-hyoscyamine. The most common atropine compound used in medicine is atropine sulfate (C₁₇H₂₃NO₃)₂.H₂SO₄.H₂O, whose full chemical name is 1-α-H, 5-α-H-Tropan-3-α ol (±)-tropate(ester), sulfate monohydrate.

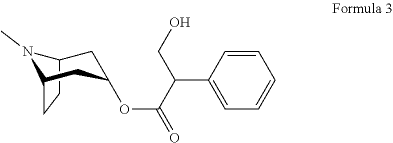

### EXAMPLE 2

### Efficacy Shown in Open Clinical Trial

Subjects were selected based on their history of experiencing performance anxiety in the context of public speaking. Subjects received oral doses of the present composition, as described below, at various times prior to speaking engagements. The parameters assessed included but were not limited to the following: tremor of voice and hands, urinary and/or fecal urgency, tachycardia, anxiety, and side effects (e.g., dry mouth).

Subject 1, a twenty-eight year old male with a history of performance anxiety ingested a combination of 40 mg propranolol HCl plus 5 mg of diphenoxylate HCl and 0.05 mg atropine sulfate on seven separate occasions and reported excellent anti-anxiety effects on all occasions. The medication was ingested 90 minutes prior to public speaking on all seven occasions. In addition, the subject reported no trembling voice or hands, no rapid heart beat and no confusion. In addition to the anti-anxiety effects, the subject reported no bowl or urinary urges. The subject reported that subsequent presentations 2 through 7 became more animated as he was able to infuse life into the presentations due to the alleviation of the symptoms of performance anxiety.

Subject 2, a fifty-six year old male with a history of performance anxiety ingested 5.0 mg diphenoxylate HCl and 0.05 mg atropine sulfate 90 minutes prior to public speaking, and reported no urinary or fecal urgency. However, the subject reported tremors of his voice and hands with a pounding heart beat that had an unfavorable effect on the delivery of the presentation. Subject 2 ingested 40 mg of propranolol HCl ninety minutes prior to public speaking. The subject reported no tachycardia or voice tremor related to the symptoms of performance anxiety, but did report fecal urgency and what he described as a less colorful and confident presentation.

Subject 2 ingested a combination of 40 mg propranolol HCl plus 5 mg of diphenoxylate HCl and 0.05 mg atropine sulfate 90 minutes prior to public speaking on five separate occasions. The subject reported no anxiety, no trembling voice, no increase in perspiration, no fidgeting, no bowel or urinary urges, increased concentration, no interfering negative thoughts, no fear and inadequate feelings. In addition, the subject reported that he felt better than normal and confident during the presentation. The subject reported insomnia the night before public speaking on occasion 1, but reported no insomnia on occasions 2 through 5 due to the medication's ability to negate the symptoms of performance anxiety. In contrast to the occasions when subject 2 ingested propranolol HCl alone, or diphenoxylate HCl and atropine sulfate alone, subject 2 reported total alleviation of performance anxiety symptoms after ingestion of propranolol HCl, diphenoxylate HCl, and atropine sulfate, and felt that the combination seemed to elevate his confidence, allowing him to be more animated, infusing life and color into the presentation. The enhanced platform skills described by subject 2 are unrelated to the known pharmacodynamic effects of propranolol HCl or of diphenoxylate HCl plus atropine sulfate, and suggests an interaction between propranolol HCl and diphenoxylate HCl, or between propranolol HCl, diphenoxylate HCl, and atropine sulfate, such that the effect when combined is greater that the predicted effect based on the response to propranolol HCl alone, or to diphenoxylate HCl plus atropine sulfate alone. In other words, the results suggest synergy between propranolol HCl and diphenoxylate HCl, or between propranolol HCl, diphenoxylate HCl, and atropine sulfate.

Subject 3, a twenty year old male with a history of performance anxiety ingested a combination of 20 mg propranolol HCl plus 2.5 mg of diphenoxylate HCl and 0.025 mg atropine sulfate two hours prior to public speaking and reported good anti-anxiety effects with no trembling voice, no fear, and no knot in his stomach. On another occasion, the subject ingested a combination of 10 mg of propranolol sulfate plus 2.5 mg of diphenoxylate HCl/0.025 mg atropine sulfate 90 minute prior to public speaking with good anti-anxiety effects.

Subject 4, a twenty-six year old male subject with a history of performance anxiety ingested a combination of 20 mg propranolol HCl plus 5 mg of diphenoxylate HCl and 0.05 mg atropine sulfate two hours prior to public speaking and reported excellent anti-anxiety effects. The subject remarked that he experienced mild dry mouth as a side effect that was alleviated with sips of water.

Subject 5, a thirty-one year old female subject with a history of performance anxiety ingested a combination of 40 mg propranolol HCl plus 5 mg of diphenoxylate HCl and 0.05 mg atropine sulfate two hours prior to public speaking. The subject reported excellent anti-anxiety effects. She reported being poised during the presentation without trembling voice, hands, arms, feet and without sudden bowl or urinary urges.

Subject 6, a twenty-one year old male college student with a history of performance anxiety ingested a combination of 40 mg propranolol HCl plus 5 mg of diphenoxylate HCl and 0.05 mg atropine sulfate 90 minutes prior to giving an introductory speech in front of 40 peers. The subject reported no trembling voice, no weak knees, no shaky hands, no confusion, no rapid heartbeat and did not “trip” over his words. The subject reported having presented his speech clearly and succinct. In addition, the subject reported that his speech was animated and natural, as he was able to focus on his speech rather than being negatively influenced by performance anxiety symptoms. The subject also commented that due to the effectiveness of the drug combination he will have no insomnia prior to his next presentation. The subject had been avoiding enrolling in a college “Speech” course due to fear of public speaking, and—following his introductory speech—framed his feelings about the combination of propranolol HCl, diphenoxylate, and atropine sulfate as “simply awesome!” The subject expressed amazement that the combination blocked his fear of public speaking completely, while also enhancing his presentation.

### EXAMPLE 3

### Large Scale Clinical Trial

The study will be an open-label, variable dose clinical trial to assess the efficacy and safety of three fixed doses of combined propranolol HCl, diphenoxylate HCl, and atropine sulfate in the treatment of performance anxiety. Up to fifty volunteers, who have given informed consent, will take either 40 mg, 20 mg, or 10 mg of propranolol HCl hydrochloride—combined with either 5 mg of diphenoxylate HCl and 0.05 mg of atropine sulfate, or 2.5 mg of diphenoxylate HCl and 0.025 mg of atropine sulfate—either once or twice over a period of two hours preceding a performance task. The task is an occasion of public speaking that the subject anticipates will be highly anxiety-producing.

Subjects will be able to vary the dose according to their need in the range of one capsule of the lowest strength up to a maximum of two capsules of the highest strength formulation. Subjects will act as their own controls and will complete a subjective evaluation of drug effects and anxiety state before and after the performance task. The results of the study are expected to verify the results of the study described in Example 1 that combined propranolol HCl, diphenoxylate HCl, and atropine sulfate is an effective treatment for reducing anxiety and enhancing performance associated with events that provoke anxiety in the patient.

Several anxiety rating scales are in common use in anxiety research (Marks et al. Behav. Res. Ther, 17:263-267, 1982; Davidson et al. J. Clin. Psych. 52(suppl):48-51, 1991). Patients will complete a series of self evaluations before and after their performance task. Scores will be computed as a percentage change from baseline on various dimensions of anxiety. Descriptive statistics will be calculated for changes in anxiety ratings between baseline and final analysis. The presence of side effects, such as dry mouth, light-headedness, nausea and difficulty urinating, will be sought specifically.

Throughout this application various publications are referenced within parentheses. The disclosures of these publications in their entireties are hereby incorporated by reference into this application in order to more fully describe the state of the art to which this disclosure pertains.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

What is claimed is:

1. A pharmaceutical composition for treating social anxiety, performance anxiety, or social phobia comprising a therapeutic amount for the treatment of a patient of a β-adrenergic receptor antagonist, an anti-diarrheal compound, and an optional anticholinergic compound.

2. The composition of claim 1, wherein the β-adrenergic receptor antagonist is propranolol HCl.

3. The composition of claim 1, wherein the anti-diarrheal compound is an opioid compound.

4. The composition of claim 1, wherein the anti-diarrheal compound is diphenoxylate HCl.

5. The composition of claim 1, wherein the optional anticholinergic compound is atropine sulfate.

6. The composition of claim 1, wherein the β-adrenergic receptor antagonist is propranolol HCl, the anti-diarrheal compound is diphenoxylate HCl, and the optional anticholinergic compound is atropine sulfate.

7. The composition of claim 6, wherein the propranolol HCl is in an amount of from about 5 mg to about 40 mg, the diphenoxylate HCl is in an amount of from about 2.5 mg to about 5.0 mg, and the optional atropine sulfate is in an amount of from about 0.025 mg to about 0.05 mg.

8. A method of treating social anxiety in a patient comprising the step of administering the composition of claim 1 to a patient in need of such treatment.

9. A method of treating social anxiety in a patient comprising the step of administering the composition of claim 6 to a patient In need of such treatment.

10. A method of preventing social anxiety in a patient comprising the step of administering the composition of claim 1 to a patient in need of such treatment.

11. A method of preventing social anxiety in a patient comprising the step of administering the composition of claim 6 to a patient in need of such treatment.

12. A method of treating performance anxiety in a patient comprising the step of administering the composition of claim 1 to a patient in need of such treatment.

13. A method of treating performance anxiety in a patient comprising the step of administering the composition of claim 6 to a patient In need of such treatment.

14. A method of preventing performance anxiety in a patient comprising the step of administering the composition of claim 1 to a patient in need of such treatment.

15. A method of preventing performance anxiety in a patient comprising the step of administering the composition of claim 6 to a patient in need of such treatment.

16. A method of treating social phobia in a patient comprising the step of administering the composition of claim 1 to a patient in need of such treatment.

17. A method of treating social phobia in a patient comprising the step of administering the composition of claim 6 to a patient in need of such treatment.

18. A method of preventing social phobia in a patient comprising the step of administering the composition of claim 1 to a patient in need of such treatment.

19. A method of preventing social phobia in a patient comprising the step of administering the composition of claim 6 to a patient in need of such treatment.

20. A method of treating social anxiety in a patient comprising the steps of:
a) administering a therapeutic amount of a β-adrenergic receptor antagonist;
b) administering, either before, after, or simultaneous to the administration of the β-adrenergic receptor antagonist, a therapeutic amount of an anti-diarrheal compound; and
c) optionally administering, either before, after, or simultaneous to the administration of the β-adrenergic receptor antagonist, a therapeutic amount of an anticholinergic compound
to a patient in need of such treatment.

21. The method of claim 20, wherein the β-adrenergic receptor antagonist is propranolol HCl, the anti-diarrheal compound is diphenoxylate HCl, and the optional anticholinergic compound is atropine sulfate.

22. A method of preventing social anxiety in a patient comprising the steps of:
a) administering a therapeutic amount of a β-adrenergic receptor antagonist;
b) administering, either before, after, or simultaneous to the administration of the β-adrenergic receptor antagonist, a therapeutic amount of an anti-diarrheal compound; and
c) optionally administering, either before, after, or simultaneous to the administration of the β-adrenergic receptor antagonist, a therapeutic amount of an anticholinergic compound
to a patient in need of such treatment.

23. The method of claim 22, wherein the β-adrenergic receptor antagonist is propranolol HCl, the anti-diarrheal compound is diphenoxylate HCl, and the optional anticholinergic compound is atropine sulfate.

24. A method of treating performance anxiety in a patient comprising the steps of:
a) administering a therapeutic amount of a β-adrenergic receptor antagonist;
b) administering, either before, after, or simultaneous to the administration of the β-adrenergic receptor antagonist, a therapeutic amount of an anti-diarrheal compound; and
c) optionally administering, either before, after, or simultaneous to the administration of the β-adrenergic receptor antagonist, a therapeutic amount of an anticholinergic compound
to a patient in need of such treatment.

25. The method of claim 24, wherein the β-adrenergic receptor antagonist is propranolol HCl, the anti-diarrheal compound is diphenoxylate HCl, and the optional anticholinergic compound is atropine sulfate.

26. A method of preventing performance anxiety in a patient comprising the steps of:
a) administering a therapeutic amount of a β-adrenergic receptor antagonist;
b) administering, either before, after, or simultaneous to the administration of the β-adrenergic receptor antagonist, a therapeutic amount of an anti-diarrheal compound; and
c) optionally administering, either before, after, or simultaneous to the administration of the β-adrenergic receptor antagonist, a therapeutic amount of an anticholinergic compound
to a patient in need of such treatment.

27. The method of claim 26, wherein the β-adrenergic receptor antagonist is propranolol HCl, the anti-diarrheal compound is diphenoxylate HCl, and the optional anticholinergic compound is atropine sulfate.

28. A method of treating social phobia in a patient comprising the steps of:
a) administering a therapeutic amount of a β-adrenergic receptor antagonist;
b) administering, either before, after, or simultaneous to the administration of the β-adrenergic receptor antagonist, a therapeutic amount of an anti-diarrheal compound; and
c) optionally administering, either before, after, or simultaneous to the administration of the β-adrenergic receptor antagonist, a therapeutic amount of an anticholinergic compound
to a patient in need of such treatment.

29. The method of claim 28, wherein the β-adrenergic receptor antagonist is propranolol HCl, the anti-diarrheal compound is diphenoxylate HCl, and the optional anticholinergic compound is atropine sulfate.

30. A method of preventing social phobia in a patient comprising the steps of:
a) administering a therapeutic amount of a β-adrenergic receptor antagonist;
b) administering, either before, after, or simultaneous to the administration of the β-adrenergic receptor antagonist, a therapeutic amount of an anti-diarrheal compound; and
c) optionally administering, either before, after, or simultaneous to the administration of the β-adrenergic receptor antagonist, a therapeutic amount of an anticholinergic compound
to a patient in need of such treatment.

31. The method of claim 30, wherein the β-adrenergic receptor antagonist is propranolol HCl, the anti-diarrheal compound is diphenoxylate HCl, and the optional anticholinergic compound is atropine sulfate.
